(19) United States
(12) Patent Application Publication
(10) Pub. No.: US 2004/0228828 A1
(43) Pub. Date: Nov. 18, 2004

(54) Odor control apparatus and methods

(76) Inventor: Mark Myrowich, Manitoba (CA)

Correspondence Address: DICKSTEIN SHAPIRO MORIN & OSHINSKY LLP, 2101 L STREET NW, WASHINGTON, DC, 20037-1526, US

(21) Appl. No.: 10/788,459

(22) Filed: Mar. 1, 2004

Related U.S. Application Data
(60) Provisional application No. 60/452,528, filed on Mar. 7, 2003.

(51) Int. Cl. A61L 9/015

(52) U.S. Cl. 424/76.2

(57) ABSTRACT

An odor control blanket having a degradable net support layer, a semi-permeable degradable protective layer, and an active layer comprising natural fibers between the support and protective layers and capable of supporting the growth of micro-organisms. The odor control blanket of the invention can be used to reduce odors associated with manure storage facilities.

### CROSS REFERENCE TO RELATED APPLICATION

This patent application claims the benefit of U.S. provisional patent application serial No. 60/452,528, filed Mar. 7, 2003 the entirety of which is hereby incorporated by reference.

### BACKGROUND OF THE INVENTION

Malodorous waste is a natural by-product of many industrial operations. Livestock producers face enormous challenges in controlling and limiting odors associated with raising and maintaining animals. Public concerns regarding odor control and the impact of waste products in general on the environment have led to an increase in scrutiny and regulation of the livestock industry. Livestock manure is very high in organic matter and nutrients and therefore has the potential to cause odor, water pollution, and contribute to greenhouse gases. A report prepared by the Hog Environmental Management Strategy Steering Committee (1997) emphasized that environmental issues, especially odors and water pollution, are among the most important factors limiting expansion of the hog industry.

Approximately 25% of odor complaints are derived from the smells emitted from manure storage facilities. Manure storage facilities, for example, are required in Canada and the Northern United States because the climate and cropping practices dictate applying fertilizers (such as manure) in the spring and fall. Manure storage is therefore necessary to retain the manure until land application is acceptable. Long term storage of manure can result in an increase in malodorous substances including a wide variety of compounds such as volatile fatty acids, aldehydes, alcohols, phenols, indoles, mercaptans, and amines.

There are various systems for storing liquid manure (slurry) including under-floor pits, outdoor storage facilities (e.g., above or below ground formed structures made of steel, concrete or fiberglass), and earthen basins. The most common type of manure storage system is the earthen storage system, the lowest cost option for manure storage. Earthen manure storage systems are generally large open-topped basins shaped from the earth and lined with clay and/or other impermeable materials. The basins provide cost effective storage for periods of up to 400 days, allowing producers to empty the facility and spread manure only once per year. Odor level from open earthen manure storage is elevated due to the large exposed surface area. The large open surface area causes a continuous source of odor especially under windy conditions. The problem becomes more severe during spring and summer months due to the increase in microbial activities. Therefore, development of cost-effective strategies to control odor would make livestock operations more attractive to rural communities and ensure the sustainability of livestock operations.

There are a number of products which profess to reduce odor from hog manure. They can be classified into the following broad categories: masking agents, deodorizing agents, bacterial and enzyme additives, solid separators, physical barriers (covers), anaerobic treatment lagoons, and aerated treatment lagoons. Many of these products act as a ‘cover-up’ to prevent the offending gases from escaping into the air. Most of these products actually do not eliminate or prevent the formation of odor-causing gases.

Anaerobic lagoons are often confused with earthen manure storages. Lagoons are carefully designed and managed to maintain optimum loading rates, retention time and temperature of the manure to maintain a balance between the acid-forming and methane-forming bacteria. Earthen manure storage structures are simply basins designed to store the manure between periods of land application. Lagoons have been used successfully in warmer climates, but in colder climates, where temperatures are low for much of the year, the methane-forming bacteria become inactive and the rate of decomposition is slow, resulting in lagoons containing unstabilized solids. During the spring when the manure temperature begins to increase, the acid-forming, rather than the methane-forming bacteria become active and begin to decompose the manure accumulated over the winter. When this occurs, the system is unbalanced and offensive odors are produced.

Straw blown on the surface of an earthen lagoon is a common, low cost physical barrier. In 1992, the Prairie Agriculture Machinery Institute (PAMI) concluded a series of five projects to develop effective odor controlling coverings for hog manure lagoons. Studies were conducted on the effectiveness of supported and unsupported covers, with emphasis on cover durability, straw type, odor reduction period, and management problems. In the final project, several straw types were used in full scale tests on lagoons. Straw was applied for both flotation supported covers and non-supported covers by using a device initially designed to spread straw for surface erosion control in road construction and by farmers to provide a surface for bedding cattle. The applicator consists of a conveyor moving square bales through a flail shredder into a paddle fan blower that blows the straw through a moveable spout. Blown straw has significant disadvantages including incomplete and shifting coverage of the lagoon, sinking of the straw, creation of problems during agitation or pump out operations, clogging of pump equipment, creating a need for use of special pumps and pressurized delivery tanks. See, e.g, U.S. Pat. No. 6,361,249, col. 1, lines 27-41.

Several types of fixed covers have been used in an attempt to reduce odors associated with waste lagoons. As shown in Table 1 below, fixed covers vary in their ability to reduce odor:

TABLE 1
Different Covers for Pig Slurry Stores and
Reduction in Emission Achieved

| Fixed Cover | Percent Emission Reduction |
|---|---|
| Chopped Straw | 70-75 |
| Granulated Layer | 80-90 |
| Floating Cover | 85-95 |
| Tent-roof construction | 90-95 |
| Solid concrete cover | 95-98 |

The least expensive cover is a floating layer of fibrous material such as chopped straw. Straw covers have become relatively commonplace across Western Canada and are often specified by local municipal governments as a condition for the approval of earthen manure storage. The Prairie Agricultural Machinery Institute [PAMI] studied the effectiveness of different natural straw covers in eliminating odors released by swine manure lagoons. The studies showed that only good quality barley straw reduced odor. Fixed covers can differ significantly in their cost. For example, while blown straw costs as little as 8 cents per square foot (including the cost of materials, labor and machinery for application of the straw), negative air pressure covers can cost between 54 cents and 1 dollar per square foot.

As an alternative, floating plastic sheets can be used to reduce gas and odor emissions. The cost of plastic covers can be much higher than straw covers. Plastic covers are susceptible to wind damage and this has prevented greater use of these covers. Plastic covers are currently being employed in some countries such as the Netherlands, but at a relatively high cost of $1.40 per square foot. Plastic covers have traditionally incorporated stabilizing panels or some form of frame, otherwise wind can displace or damage the cover. For larger surfaces, a tent with a central pillar support, covered by high quality plastic material with textile reinforcement is used. These are especially popular in the Netherlands. Koerner (1994) reported that fixed covers may not be suitable for structures with a span greater than 15 meters. However, wind can exert large forces that push the cover aside or result in tears in the material. Plastic covers have not seen widespread adoption by the livestock industry because of their high costs. Traditional plastic covers must be durable since these covers must withstand the forces imposed by high winds, snow, and rain. Previously available plastic covers were designed to remain on manure storage facilities for extended periods of time (e.g., greater than one year).

The longevity and durability of plastic covers may also be limited by exposure to ultraviolet (UV) light from the sun. UV light can breakdown materials used to form plastic covers, such as polypropylene. Therefore, previously available plastic covers were designed to be resistant to degradation. For example, carbon black (e.g., Kepital Copolymer and Lucet Copolymer available from K-Mac Plastics Kentwood, Mich. 49518) may be added to plastic to increase its resistance to degradation. U.S. Pat. No. 5,221,568 (“the '568 patent”) relates to a water and/or oil-impermeable sealing mat comprising a substrate layer and a cover layer made of a non-woven textile material, and an intermediate layer of swellable clay. The non-woven fabrics and plastics used in the sealing mats are “resistant to rotting (resistant to all substances occurring in bodies of water and in soil) and thus warrant evidently an extremely long service life . . . [t]hey are highly stable towards ultra-violet radiation.” '568 patent, col. 7, lines 28-34. U.S. patent application 2002/0139742 (“the '742 application”) refers to a lagoon cover having flotation panels formed from bonded foam particles. According to the '742 application, “the waste confinement area covers of the present invention provide long life expectancies, are UV resistant, impervious to most chemicals, and retain their properties in temperatures ranging from about −50° C. to about 55° C. '742 application, page 7, paragraph 67. U.S. patent application 2002/0192031 (“the '031 application”) relates to a cover assembly for organic waste lagoon basins made of geotextile materials that are stabilized to resist degradation due to ultraviolet radiation exposure. The '031 application is said to be “fabricated from a geotextile material that can be polypropylene fiber, non-woven, needle punch fabric stabilized to resist degradation due to ultraviolet light exposure.” '031 application, page 1, paragraph 4.

What is needed are cost-effective and environmentally-friendly products and processes for controlling odor from earthen lagoons, for storing livestock manure and similar odor sources encountered in the livestock and related industries.

### BRIEF SUMMARY OF THE INVENTION

The invention relates to an odor control blanket including a degradable net support layer, a semi-permeable degradable protective layer, and an active layer of natural fiber straw (e.g., barley straw). The odor control blanket is capable of supporting the growth of micro-organisms and is capable of floating on top of a liquid or semi-solid odorous source such as an animal waste lagoon.

In one embodiment, the active layer is “sandwiched” between the support layer and the protective layer. The three layers may be stitched together by, for example, needles carrying thread through each layer. The stitching process can also perforate the layers, making the odor control blanket semi-permeable. The invention also relates to methods of reducing odors associated with animal waste by applying an odor control blanket having a protective layer, an active layer, and a support layer to the surface of a waste basin for a period of about one to two years. The blanket may then be removed and replaced with another odor control blanket.

Additional embodiments and advantages of the present invention will be set forth in the description that follows and will be obvious from the description, or may be learned through the practice of the invention. The objects and advantages of the invention will be attained by means of the instrumentalities and combinations particularly pointed out in the appended claims.

### BRIEF DESCRIPTION OF THE DRAWINGS

FIG. 1 shows an exemplary odor control blanket having a degradable, semi-permeable, protective layer 1, an active layer 2, and a net support layer 3 in accordance with a preferred embodiment of the invention.

FIG. 2 shows an elevated view of an exemplary odor control blanket depicting examples of stitching and perforations 4 on the protective layer 1.

### DETAILED DESCRIPTION OF THE INVENTION

An effective and practical solution for odor control of manure storage facilities should balance the cost of the covering with its effectiveness in reducing odors. A covering which reduces 100% of the odors associated with manure storage but is cost prohibitive will not provide a practical, widespread, and economical solution to the problem. While blown straw is a relatively inexpensive covering it has significant drawbacks, including incomplete covering, susceptibility to wind, clogging of manure storage “clean-out” pumps and hoses, and increased labor costs for application and reapplication of the covering. Negative air pressure coverings, while effective for odor control, can be cost prohibitive and require additional maintenance. The odor control blankets of the invention, in contrast, combine a bioactive natural covering with a plastic covering. Furthermore, the odor control blankets of the invention are degradable in order to breakdown naturally. Unlike previously available coverings, they are not required to have increased resistance to wind, rain, and sunlight. The degradable odor control blankets of the invention are therefore easier to dispose of and more environmentally friendly.

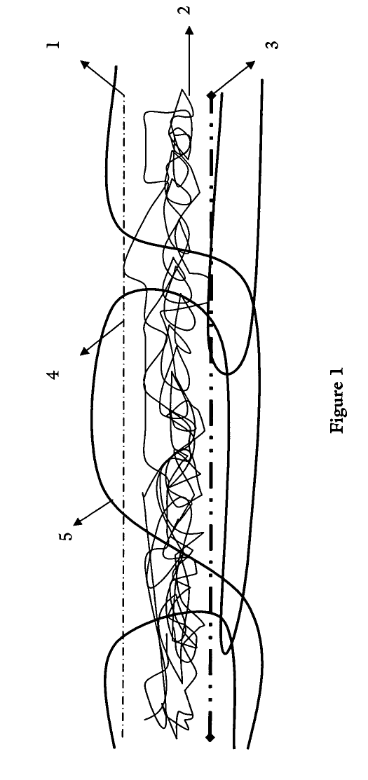

As shown in FIG. 1, one embodiment of the invention provides an odor control blanket comprising a degradable net support layer 3, a semi-permeable degradable protective layer 1, and an active, degradable, layer 2 comprising natural fibers. The layers are preferably sewn together with thread 5.

An exemplary net support layer 3 is formed from polypropylene and provides a surface upon which natural fibers are spread. The net support layer 3, preferably, has openings large enough to permit liquids to pass through yet small enough to provide adequate support to the natural fibers (i.e., the natural fibers do not fall through the openings). For example, the openings can be from about 1 to 25 inches in diameter.

A preferred degradable protective layer 1 is formed from degradable polypropylene which is subject to natural degradation over time. The term “degradable” refers to the ability of the material to breakdown over time due to environmental conditions (e.g., biodegradation by microorganisms, photo degradation by UV light). In another embodiment of the invention, the degradable protective layer 1 is perforated 4 in order to make the layer semi-permeable (e.g., permit the exchange of gases and water above and below the protective layer). Alternatively, the protective and support layers can be formed from the following degradable materials: jute matting and netting, canvas, coir matting and netting, cotton fabric and netting , rayon fabric, polyester fabric, polyethylene plastic, paper, and needle punched or woven polypropylene fibers or strands (e.g., geotextile material). The protective and support layers may be formed at any suitable thickness (e.g., about 1/8 inch to 1 inch thick). The preferred materials used to form the protective layer do not include additives to increase resistance to degradation (e.g., carbon black).

An exemplary active layer 2 is formed from natural fibers which are capable of supporting the growth of microorganisms. Natural fibers include, but are not limited to, barley straw, wheat straw, oat straw, rice straw, flax straw, coir fiber (coconut), wood fiber (excelsior fiber), hemp fiber, and cotton. Without wishing to be bound by theory, it is believed that microorganism growth in and around the active layer 2 reduces odors (e.g., by metabolizing volatile fatty acids). Thus, the odor reducing features of the UV degradable polypropylene support layer and protective layer are enhanced by the odor reducing properties of the natural fibers in the active layer.

The three layers (degradable protective layer, active layer, and the degradable support layer) are preferably attached to each other forming a multi-layered odor control blanket. In another embodiment of the invention, the three layers are stitched together by threading an attachment material (e.g., thread) through all three layers. The attachment material can be made of any suitable thread material (e.g., jute thread, canvas, coir thread, cotton thread, rayon thread, polyester thread, polyethylene plastic thread, paper thread, and polypropylene thread). The strength of the preferred thread is between about 400 and 1200 deniers. In a preferred embodiment, the strength of thread is about 800 deniers. The odor control blankets can be of any suitable width (e.g., 5 to 24 feet wide) and length (e.g., 50 to 600 ft long) and can be custom made to cover a manure waste facility of any size or shape. In a preferred embodiment, the odor control blanket is about 16 feet wide and 250 feet in length.

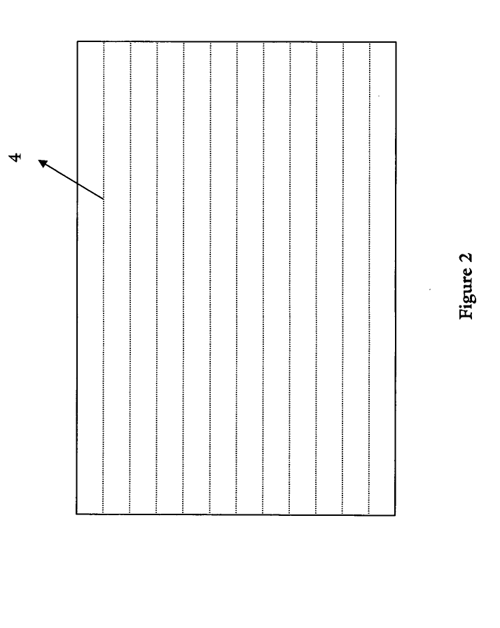

Preferably, the thread material is threaded through perforations made in the degradable protective cover and around the net in the support layer. In one embodiment of the invention, the holes in the protective layer are made with needles forming a continuous thread trail running the length of the blanket with each trail spaced about 1 to 2 inches apart from each other. The thread is pulled through using needles in a chain link pattern about 2 and 4 inches in length of each “link”. As the needles stitch the three layers together, they perforate the layers and draw the thread through all three layers from hole to hole. In another embodiment of the invention, the protective layer is preferably perforated and stitched to the other layers laterally across the entire length of the blanket with perforations uniformly spaced apart (e.g., about 1 to 2 inches). An exemplary perforation 4 and stitching pattern is depicted in FIG. 2. Alternatively, the protective layer can be perforated and stitched to the other layers in any suitable pattern in order to attach the layers to each other and form a semi-permeable protective layer.

In a preferred embodiment of the invention, the odor control blanket is buoyant and capable of being placed on the surface of liquid or solid waste or waste containing both liquids and solids. The odor control blanket can, for example, be rolled out over the surface of a manure waste facility. Another embodiment of the invention provides a method of reducing odors from an odor source by placing an odor control blanket including a UV degradable net support layer, a semi-permeable UV degradable protective layer, and an active layer of natural fibers on an odor source for a period of about one to two years, removing the odor control blanket, and replacing the odor control blanket with at least a second odor control blanket.

In one embodiment of the invention, an odor control blanket can be manufactured by the following exemplary method. Natural fiber in bale form is placed in a bale buster to loosen the fiber by beating the bale with flails. The loosened fiber is then pneumatically blown into a fleecing box which feeds a tambour (fleecing area). The net support layer (which may be provided in large rolls) is unrolled between the bale buster and the fleecing box and the net support layer is pulled under the fleecing box and tambour. The fiber is pulled through rollers and feeders from the fleecing box to the tambour which spreads the natural fiber out evenly over the entire width of the net support layer. The net support layer is pulled by rollers past the tambour and fleece box where the protective layer is placed over the bottom two layers. The three layers are then pulled by roller to a stitching bar which stitches thread through the three layers in a chain link fashion. The needles are in rows perpendicular to the machine about 1.5 to 2 inches apart from each other. The needles can sew a chain link between about 2 and 4 inches in length. As the needles stitch the three layers together, they perforate the layers and draw the thread through all three layers from hole to hole. Once past the stitching bar, the odor control blanket is then rolled into 50 to about 600 foot length rolls and packaged.

The odor control blankets reduce odors associated with an odor source more effectively than blown straw alone. The odor control blankets of the invention prevent the natural fiber active layer from sinking and thus avoid problems associated with blown straw in manure waste facilities. In addition, the odor control blankets of the invention immobilize the natural fiber active layer and therefore provide a more uniform cover of the natural fiber across the surface of the manure waste facility. The combination of an active layer and a UV degradable plastic cover is more effective in reducing odors than a plastic cover alone. It is believed that the active layer supports the growth of microorganisms that reduce odors. Furthermore, the layered structure of the odor control blankets of the invention provides a moist, humid environment that encourages additional microorganism growth. The semi-permeable protective layer permits the exchange of gas and liquids through the layers of the odor control blanket further facilitating microorganism growth while maintaining the buoyancy of the odor control blanket.

The odor control blankets of the invention are more cost effective than negative air pressure covers. In addition, negative air pressure covers require additional machinery and maintenance. Also, the odor control blankets of the invention are highly degradable making them more environmentally friendly. Although blown straw might initially appear to be less expensive than the odor control blankets of the invention, application of blown straw requires specialized machinery and additional labor to cover a manure storage facility. Often, reapplication of the straw is necessary to ensure proper coverage of the facility. In addition, there are additional costs associated with pumping out the waste facility and the potential for damage to the pump caused by clogged straw. In contrast, the odor control blankets of the invention can simply be rolled out over the manure storage facility. The natural fiber is held between the protective and support layers and therefore will not damage the pump.

It is to be understood that application of the teachings of the present invention to a specific problem or environment will be within the capability of one having ordinary skill in the art in light of the teachings contained herein.

What is claimed as new and desired to be protected by Letters Patent of the United States is:

1. An odor control blanket comprising a degradable net support layer, a semi-permeable degradable protective layer, and an active layer comprising natural fiber straw, wherein the layers are attached to each other and the active layer is capable of supporting the growth of micro-organisms.

2. A method of reducing odors from an odor source comprising placing an odor control blanket comprising a degradable net support layer, a semi-permeable degradable protective layer, and an active layer comprising natural fibers on an odor source for a period of about one to two years, removing the odor control blanket, and replacing the odor control blanket with at least a second odor control blanket.
